(19) United States
(12) Patent Application Publication
(10) Pub. No.: US 2008/0255532 A1
(43) Pub. Date: Oct. 16, 2008

(54) Multiple barrier leg cuff

(75) Inventor: Charles F. Schroer, Camden, AR (US)

Correspondence Address: DICKSTEIN SHAPIRO LLP, 1177 AVENUE OF THE AMERICAS (6TH AVENUE), NEW YORK, NY, 10036-2714, US

(73) Assignee: ARQUEST, INC., Cranbury, NJ (US)

(21) Appl. No.: 11/783,859

(22) Filed: Apr. 12, 2007

(51) Int. Cl. A61F 13/15

(52) U.S. Cl. 604/385.26; 604/385.25

(57) ABSTRACT

An absorbent article having a liquid permeable topsheet, a liquid impermeable backsheet, an absorbent core disposed in between, and multiple barrier leg cuffs. The multiple barrier leg cuffs have at least one elasticized cuff and at least one non-elasticized cuff.

### FIELD OF INVENTION

The present invention relates to absorbent articles such as diapers, training pants, and incontinence briefs. More particularly, the present invention relates to absorbent articles having multiple barrier leg cuffs for the absorption and containment of body exudates.

### BACKGROUND OF INVENTION

Absorbent articles are well-known in the art. Typically, such articles comprise a liquid permeable body-contacting top sheet, a liquid impermeable back sheet, a moisture absorbent core that is generally disposed between the topsheet and backsheet, a barrier cuff, and a liquid impermeable gasketing cuff. These absorbent articles often include additional features such as elastic waist bands, elastic leg bands, and stretchable side panels. In addition, decorative graphics are often incorporated into the absorbent article for aesthetic and functional purposes.

The major function of absorbent articles such as infant diapers or training pants, adult incontinence products, and other such products is to absorb and contain body exudates. Such articles are thus intended to prevent body exudates from soiling, wetting, or otherwise contaminating clothing or other articles, such as bedding, that come in contact with the wearer. One common mode of failure for such products occurs when body exudates leak out of the gaps between the article and the wearer's leg to adjacent clothing because they are not immediately absorbed within the articles.

Manufacturers have used elasticized gasketing cuffs to prevent wicking and overflow of liquid from a fluid laden absorbent article to clothing contacting the edges of the article. The gasketing cuff presents a barrier between the edge of the article and any adjacent clothing. In addition, the elasticized gasketing cuff provide a gasketing action about the legs of the wearer. However, the use of the gasketing cuff alone as a containment means is often ineffective, particularly in the case of subsequent insults which take longer to be absorbed due to a partially saturated absorbent core.

Elasticized barrier cuffs have been added to absorbent articles to compliment gasketing cuffs. The barrier cuffs are typically positioned laterally inboard of the gasketing cuffs. The barrier cuffs assist in inhibiting loose fecal matter or gushes of urine or liquids from soiling the wearer's clothing. The use of barrier cuffs is not always effective in restraining the lateral flow of bodily waste. Consequently, to the extent that bodily waste migrates over the top of the barrier cuffs, conventional diapers are susceptible to leakage beyond their last line of defense, the gasketing cuffs.

In response to attempts to control the lateral flow of bodily waste, the prior art has incorporated elasticized dual barrier cuff assemblies onto each side of a single absorbent article. See for example, U.S. Pat. No. 5,167,653 and U.S. Stat. Inv. Reg. H1630. While providing elasticized inner and outer cuffs within an absorbent article has its advantages, the resulting article is often bulky in the crotch area, thus causing discomfort to the wearer. Therefore, what is needed is an absorbent article that provides both effective control of leakage and comfort in the crotch region.

### SUMMARY OF THE INVENTION

In response to the difficulties encountered in the prior art, the present invention provides an improved absorbent article having at least one elasticized barrier leg cuff and at least one non-elasticized barrier cuff.

The absorbent article of the present invention preferably includes a topsheet, a backsheet, an absorbent core positioned between the topsheet and backsheet, and gasketing cuffs located inboard of respective side edges of the absorbent article. Multiple barrier leg cuffs are preferably positioned along the topsheet inboard of the gasketing cuffs. The multiple barrier leg cuffs include at least one non-elasticized barrier leg cuff and at least one elasticized barrier leg cuff. The elasticized barrier leg cuff envelopes at least one elastic, which is secured to the barrier leg cuff near its ends or along its entire length.

The multiple barrier leg cuffs define a containment region that is useful in containing insults, and when necessary, subsequent insults that generally take longer to be absorbed due to a partially saturated core. By having multiple barrier leg cuffs attached along the topsheet inboard of the gasketing cuffs, the assembly advantageously provides a greater barrier against the lateral flow of body waste than gasketing cuffs alone or gasketing cuffs and a single barrier leg cuff. Moreover, since the multiple barrier leg cuffs of the present invention do not increase the amount of elastic in the crotch region of the absorbent article, comfort for the wearer is also increased compared to dual elasticized barrier leg cuffs.

With the present invention, to the extent that any subsequent insults migrate over the top of the first of the multiple barrier leg cuffs, the containment region created between the multiple barrier leg cuffs provides another path for acquiring bodily waste into the absorbent core. This containment region provides additional protection, which may be needed when multiple, explosive insults cannot be absorbed by the absorbent core fast enough, resulting in a lateral flow of exudates towards the sides of the absorbent article.

In addition to being comfortable and effective, absorbent articles in accordance with present invention having elasticized and non-elasticized barrier leg cuffs are economical to manufacture. For example, the cost of materials is reduced because at least one of the cuffs does not contain elastics. Further, the elimination of elastics in at least one of the barrier leg cuffs saves manufacturing time during production by removing a step in the assembly process.

These and other objects, features, and advantages of the invention will be apparent through the detailed description of the preferred embodiments and the drawings attached hereto. It is also understood that both the foregoing general description and the following detailed description are exemplary and explanatory and are not restrictive of the scope of the invention.

### BRIEF DESCRIPTION OF THE DRAWINGS

The present invention will be more fully understood and further advantages will become apparent when reference is made to the following detailed description of the invention and the drawings, wherein:

FIG. 1 is a top plan view of an absorbent article having at least one elasticized barrier leg cuff and at least one non-elasticized barrier leg cuff, according to an embodiment of the invention; and

FIG. 2 is a cross-sectional representation of the absorbent article of FIG. 1 along line A-A.

### DETAILED DESCRIPTION OF THE INVENTION

As used herein, the term “absorbent article” refers to articles which absorb and contain liquids and more specifically refers to articles that are placed against or in proximity to the body of the wearer to absorb and contain the various exudates discharged form the body. Absorbent articles are typically differentiated by whether they are reusable or disposable. “Disposable” refers to articles which are intended to be discarded after a single use (i.e. they are not intended to be laundered or otherwise restored or reused.) The absorbent articles of the present invention are preferably disposable absorbent articles.

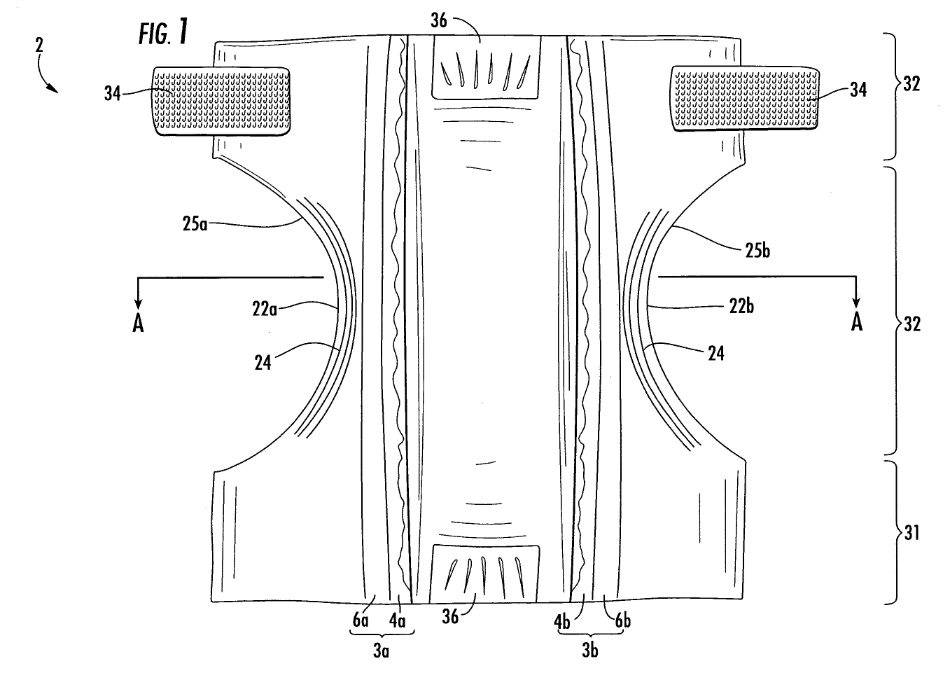

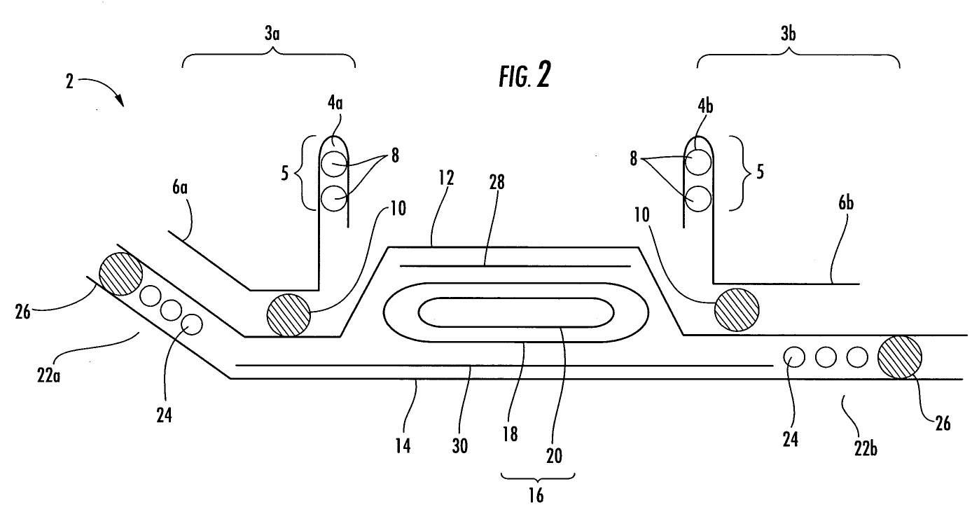

Referring now to the drawings, a preferred embodiment of the present invention is described with reference to FIGS. 1 and 2. It should be noted that the drawing figures are not to scale and certain features shown therein have been enhanced or enlarged for purposes of illustration only. As shown in FIG. 1, the absorbent article 2 has a front waist portion 31, a rear waste portion 32, and a crotch portion 33. Elastic waist elements 36 are shown extending across the top edges of the front waist portion 31 and rear waist portion 32. If the absorbent article is formed into a diaper, it may also include a fastening system 34 to hold the absorbent article 2 on the wearer. The fastening system 34 can comprise tape tabs, hook and loop fastening components, interlocking fasteners such as tabs and slots, buckles, buttons, snaps, and/or any other suitable fastening means.

As shown in FIG. 2 an absorbent core 16 is positioned between a liquid impermeable backsheet 14 and a liquid permeable topsheet 12. The absorbent core 16 may include a pulp component 20, preferably comprising cellulostic fibers, such as comminuted softwood pulp fibers. The pulp component 20 may also be mixed with distributed of a superabsorbent polymer (SAP), with the resulting pulp core 20 being preferably surrounded by a tissue core wrap 18 or other known absorbent material in order to contain the pulp 20. It should be recognized that the absorbent core 16 may be comprised of any absorbent material or materials known in the art and accordingly should not be limited to the description above. Further, the absorbent core 16 may extend the entire length of the absorbent article, or may extend only partially along the entire length of the absorbent article. The shape of the absorbent core 16 may be rectangular, hourglass, T-shaped, or any other desired shape.

An acquisition layer 28 may optionally be included adjacent to the absorbent core 16, if so desired. The acquisition layer 28 may preferably be comprised of any material having a wicking ability, and may be designed to cover only a portion of the surface of the absorbent core 16. Alternatively, it may extend across the entire surface of the absorbent core 16. The acquisition layer 28 is used to acquiesce fluid from the topsheet to the absorbent core.

The liquid permeable topsheet 12 is preferably made from any suitable material known in the art, including polymeric fabrics such as polyolefin non-woven fabrics. Common polyolefin non-woven fabrics include polypropylene and polyethylene spunbound fabrics. Additionally, the top sheet may also be formed from non-woven bicomponent polymeric fabrics. The liquid permeable topsheet 12 may also be surface treated to render it hydrophilic.

As illustrated in FIG. 2, the topsheet 12 is preferably attached to the top surface of the backsheet 14 by a suitable affixing element 26 such as an adhesive or ultrasonic or thermal bonding techniques. It should be recognized, however, that the topsheet 12 may also be attached to the top surface of the absorbent core 16.

In the preferred embodiment shown in FIG. 2, the backsheet 14 includes an outer layer 29 and a barrier layer 30 adjacent an inner surface of the outer layer 29. The outer layer 29 and barrier layer 30 are preferably laminated, glued or otherwise attached together to form the backsheet 14 of the absorbent article 2. Preferably, the outer layer 29 is formed from a liquid permeable, non-woven material, and the barrier layer 30 is formed from a liquid impermeable poly sheet material. In alternate embodiments, the backsheet can be a single piece of liquid impermeable material, or multiple layers of permeable/non-permeable material, the choice of construction being left to the designer of the absorbent article.

In addition, gasketing cuffs 22a, 22b are located in leg opening regions inboard of respective side edges 25a, 25b of the absorbent article 2. The gasketing cuffs 22a, 22b are formed by locating elastics 24 between the topsheet 12 and backsheet 14, shown in FIG. 2. The elastics 24 preferably extend through the length of the leg opening regions to provide a form-fitting gasketing cuff around the legs of the wearer of the absorbent article.

Multiple barrier leg cuffs 3a, 3b are joined to the top surface of the liquid permeable topsheet 12. Preferably, each leg cuff 3a, 3b is positioned between the absorbent core 16 and the gasketing cuffs 22a, 22b. Multiple barrier leg cuffs 3a, 3b may be joined to the topsheet using a suitable affixing element 10 such as an adhesive or ultrasonic or thermal bonding techniques. As bodily fluid is discharged, the multiple barrier leg cuffs 3a, 3b contain and hold the discharged fluid for a time sufficient to permit the fluid to be captured by the absorbent core 16.

The multiple barrier leg cuffs 3a, 3b include at least one elasticized barrier leg cuff 4a, 4b and at least one non-elasticized barrier leg cuff 6a, 6b. Preferably, the non-elasticized barrier leg cuffs 6a, 6b extend from the elasticized barrier cuffs 4a, 4b such that a continuous cuff material is used to form the multiple barrier leg cuffs 3a, 3b. In the preferred embodiment, the elasticized barrier leg cuffs 4a, 4b and the non-elasticized barrier leg cuffs 6a, 6b are formed from a liquid impermeable, nonwoven material. In addition, and to simplify assembly of the absorbent article, it is preferred that the multiple barrier leg cuffs 3a, 3b be formed from a continuous cuff material as shown in FIGS. 1 and 2. It is possible, however, to form the multiple barrier leg cuffs 3a, 3b from separate and/or different cuff materials.

The elasticized barrier leg cuffs 4a, 4b are preferably formed by enveloping elastics 8 between the folded envelope 5 of the elasticized barrier leg cuffs 4a, 4b. The elastics 8 preferably extend parallel to one another within the folded envelope 5 of the elasticized barrier leg cuffs 4a, 4b and may exhibit different degrees of extensibility depending upon their location within the elasticized barrier leg cuff 4a, 4b. For instance, the elastics 8 closest to the absorbent core may exhibit a higher degree of elasticity than the elastics 8 further from the absorbent core. The converse construction is also within the scope of the preferred embodiment.

As shown in FIG. 1, the multiple barrier leg cuffs 3a, 3b preferably extend along the entire length of the topsheet 12. Alternately, the multiple barrier leg cuffs 3a, 3b may extend only partially along the length of the topsheet 12.

Although the present invention has been described in relation to a diaper 38, it should be understood that the present invention is also applicable, but not limited to, other disposable absorbent articles such as incontinence briefs, training pants, belted diapers, diaper holders, sanitary napkins and the like.

While the invention has now been described with reference to several preferred embodiments and illustrated with regard to a range of optional features, those skilled in the art will appreciate the various substitutions, omissions, modifications and changes may be made without departing from the spirit hereof. Accordingly, it is intended that the foregoing descriptions be deemed merely exemplary of the preferred scope of the present invention and not to be deemed a limitation thereof.

What is claimed is:

1. An absorbent article comprising:
an absorbent core having a garment surface and a body surface;
a liquid impermeable backsheet adjacent to the garment surface of the absorbent core;
a liquid permeable topsheet adjacent to the body surface of the absorbent core;
at least one elastic barrier leg cuff positioned along the topsheet; and
at least one non-elastic barrier leg cuff positioned along the topsheet and adjacent to the at least one elastic barrier leg cuff.

2. The absorbent article of claim 1, wherein the at least one elastic barrier leg cuff is positioned between the absorbent core and the at least one non-elastic barrier leg cuff.

3. The absorbent article of claim 1, wherein the at least one elastic barrier leg cuff is comprised of a nonwoven material.

4. The absorbent article of claim 3, wherein the at least one elastic barrier leg cuff is comprised of a liquid impermeable material.

5. The absorbent article of claim 1, wherein the at least one non-elastic barrier leg cuff is comprised of a nonwoven material.

6. The absorbent article of claim 5, wherein the at least one non-elastic barrier leg cuff is comprised of a liquid impermeable material.

7. The absorbent article of claim 1, further comprising a gasketing cuff positioned inboard from a side edge of the absorbent article.

8. The absorbent article of claim 7, wherein the at least one non-elastic barrier leg cuff is positioned between the gasketing cuff and the at least one elastic barrier leg cuff.

9. An absorbent article comprising:
an absorbent core having a garment surface and a body surface;
a liquid impermeable backsheet adjacent to the garment surface of the absorbent core;
a liquid permeable topsheet adjacent to the body surface of the absorbent core; and
a continuous cuff material positioned along the topsheet, the continuous cuff material having at least one elastic barrier leg cuff and at least one non-elastic barrier leg cuff.

10. The absorbent article of claim 9, wherein the at least one elastic barrier leg cuff is positioned between the absorbent core and at least one non-elastic barrier leg cuff.

11. The absorbent article of claim 9, wherein the continuous cuff material is comprised of a nonwoven material.

12. The absorbent article of claim 11, wherein the continuous cuff material is comprised of a liquid impermeable material.

13. The absorbent article of claim 9, further comprising a gasketing cuff positioned inboard from a side edge of the absorbent article.

14. The absorbent article of claim 13, wherein the at least one non-elastic barrier leg cuff is positioned between the gasketing cuff and the at least one elastic barrier leg cuff.

15. An absorbent article comprising:
an absorbent core having a garment surface and a body surface;
a liquid impermeable backsheet adjacent to the garment surface of the absorbent core;
a liquid permeable topsheet adjacent to the body surface of the absorbent core;
at least one elastic barrier leg cuff positioned adjacent to the absorbent core;
at least one non-elastic barrier leg cuff adjacent to the elastic barrier cuff; and
a gasketing cuff positioned adjacent to the at least one non-elastic barrier leg cuff.

16. The absorbent article of claim 15, wherein the at least one elastic barrier leg cuff is comprised of a nonwoven material.

17. The absorbent article of claim 16, wherein the at least one elastic barrier leg cuff is comprised of a liquid impermeable material.

18. The absorbent article of claim 15, wherein the at least one non-elastic barrier leg cuff is comprised of a nonwoven material.

19. The absorbent article of claim 15, wherein the at least one non-elastic barrier leg cuff is comprised of a liquid impermeable material.

20. The absorbent article of claim 15, wherein the at least one non-elastic barrier leg cuff is positioned between the gasketing cuff and the at least one elastic barrier leg cuff.
